(19) United States
(12) Patent Application Publication
(10) Pub. No.: US 2002/0183279 A1
(43) Pub. Date: Dec. 5, 2002

(54) Pharmaceutical composition containing viscoelastic substance and medication

(75) Inventor: Koichiro Tanaka, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: Koichiro TANAKA, Tokyo (JP)

(21) Appl. No.: 10/096,715

(22) Filed: Mar. 13, 2002

(30) Foreign Application Priority Data
Apr. 18, 2001 (JP) 2001-120001

(51) Int. Cl. A61K 31/737; A61K 31/724

(52) U.S. Cl. 514/54

(57) ABSTRACT

The present invention provides a pharmaceutical composition for preventing and/or treating an infections and inflammations associated with intraocular surgery and intra-articular injection. The present invention is a pharmaceutical composition containing a medication and a viscoelastic substance.

### CROSS-REFERENCE TO RELATED APPLICATIONS

This application is based upon and claims the benefit of priority from the prior Japanese Patent Application No. 2001-120001, filed Apr. 18, 2001, the entire contents of which are incorporated herein by reference.

### BACKGROUND OF THE INVENTION

1. Field of the Invention

The present invention relates to a pharmaceutical composition for preventing and/or treating infections and inflammations associated with intraocular surgery and intra-articular injection.

2. Description of the Related Art

Viscoelastic substances such as sodium hyaluronate and chondroitin sulfuric acid are now frequently used in the clinical treatment to prevent the space of a body cavity from collapsing, protect the inner surface of the cavity and reduce friction. In particular, viscoelastic substances are indispensable medications in ophthalmic surgery, especially surgery applied to the anterior chamber, more particularly, cataract surgery.

For instance, in cataract surgery involving the insertion of an artificial lens into the eye, a viscoelastic substance is used to protect the anterior chamber from collapsing. Also it has recently become to use a viscoelastic substance to prevent the anterior chamber from collapsing when an incision is made in the anterior capsule and to protect the endothelium of the cornea during ultrasonic emulsification suction [Japanese Journal of Ophthalmic Surgery, p 209-214, Vol. 13, No. 2, 2000]. A viscoelastic substance is injected into the anterior chamber or lens capsule in almost all cases of cataract surgery.

Since a viscoelastic substance helps to protect the space of the intraocular cavity from collapsing, the surgery can be performed more accurately and safely. Cataract surgery without the use of a viscoelastic substance is unimaginable. On the other hand, a new method (called the “soft shell technique”) of using a low-molecular weight viscoelastic substance having a surface-protecting function has been established for protecting the endothelium of the cornea.

Viscoelastic substances are also used in orthopedic treatment for articular disorders. For example, articular osteoarthritis, shoulder joint periarthritis, and chronic rheumatism can be treated by injecting a viscoelastic substance into the joint space.

When a viscoelastic substance is used in surgery involving an aseptic body cavity such as the intraocular space and the joint space, it may cause bacterial contamination of the cavity. Also, in the case of the intraocular space, once the viscoelastic substance is introduced, traces of it remain within the intraocular space after surgery and is believed to cause inflammation or increase ocular tension.

The surgery of the anterior chamber is completed after the applied viscoelastic substance is removed from the eye by suction. However, it is impossible to remove all traces of viscoelastic substance from the eye. According to the reports of pharmaceutical manufacturers, traces of the viscoelastic substance remain for a certain time in the anterior chamber. The viscoelastic substance left in the anterior chamber is slightly decomposed by intraocular enzymes but mostly discharged outside the eye along with the aqueous humor flowing out from the anterior chamber. For example, a half-life of a protective agent, Viscoat (trade name), which has been approved under No. 21100 AMY 00233000, is approximately 4 hours in the anterior chamber after its injection into the eyeball.

Conventionally, when post-operative infection is likely to occur after operation is performed by introducing a viscoelastic substance into an aseptic cavity, an antibacterial agent is administered. For instance, an antibacterial agent is systemically introduced into the eyeball by administering it orally or intravenously, or locally by an ophthalmic solution.

After cataract surgery, complications may occur, the most serious being endophthalmitis. If endophthalmitis occurs in a patient, various antimicobial therapies, for example, with eye drops, by oral administration, intravenous injection and intraocular injection and the like, can be selected to treat the patient. However, once endophthalmitis appears, resistance to the anti microbial agent thus introduced may develop. If this happens, vitreum surgery (intraocular quarantine and cleaning) must be performed. Of patients that recover from endophthalmitis, few attain the eyesight of the level predicted before the cataract surgery. In some cases, the eyesight becomes worse than before the surgery, and in the worst case, a patient will become blind.

As described above, if treatment such as oral administration, intravenous injection, or external administration by eye drops is applied, normal flora may be disturbed, generating resistant strains. This phenomenon is believed to occur at a high rate, and thus a matter of great concern in hospitals and clinics all over the world.

Furthermore, a viscoelastic substance introduced into the body enfolds bacteria and keeps them from in vivo biological attacks such as disinfection and sterilization.

### BRIEF SUMMARY OF THE INVENTION

The present invention has been attained in view of the aforementioned circumstances. An object of the present invention is to provide a pharmaceutical composition capable of preventing and/or treating bacterial infection and/or inflammation in surgery such as intraocular surgery and intra-arthritis surgery performed by applying a viscoelastic substance to an aseptic cavity.

The present inventors have conducted intensive and extensive studies to attain the object. As a result, they found the following means, that is, a pharmaceutical composition containing a medication and a viscoelastic substance.

### BRIEF DESCRIPTION OF THE SEVERAL VIEWS OF THE DRAWING

FIG. 1 is a graph showing a proliferation curve of Staphylococcus aureus;

FIG. 2 is a graph showing a proliferation curve of Stenotrophomonas maltophilia;

FIG. 3 is a graph showing the inhibition circle of a viscoelastic substance against Bacillus subtilis;

FIG. 4 is a graph showing the relationship between the diameter of the LVFX(levofloxacin) inhibition circle against Bacillus subtilis and the concentration of a medication; and

FIG. 5 is a graph showing the relationship between the diameter of the NFLX(norfloxacin) inhibition circle against Bacillus subtilis and the concentration of a medication.

### DETAILED DESCRIPTION OF THE INVENTION

According to an aspect of the present invention, there is provided a pharmaceutical composition containing a medication and a viscoelastic substance. The present inventors found that a viscoelastic substance may be responsible for the deposition and proliferation of bacteria invaded into a body cavity. Based on such a finding, the present invention has been attained.

The present inventors experimentally monitored the on-set process of endophthalmitis and elucidated the on-set mechanism. More specifically, they performed experiments in-vitro using an endophthalmitis rabbit model. As a result, although the degree of inflammation significantly varies depending upon the type and the number of bacterial cells, it was observed that the inflammation of endophthalmitis spread along the passage of the aqueous humor discharged from the anterior chamber. This observation suggests that bacteria and bacterial toxin may be discharged through this passage. The passage of aqueous humor from the anterior chamber starts from the anterior chamber reaches an aqueous vein through the gonion, anterior chamber trabecula, the canal of Schelemm, and a collecting tube.

Bacteria were found in the vitreum in few experimental models. The anterior chamber was filled with pus in the case of serious inflammation. However, usually, bacteria were deposited and proliferated along the anterior surface of the iris. This fact means that, clinically, culture derived from the intraoculer is rarely obtained from a specimen taken from the vitreum and the aqueous humor from the anterior chamber. This fact conversely means the possibility that bacteria may present in the inner surface of the eyeball cavity (e.g., the anterior surface of iris), even if bacteria are not clinically obtained from the specimen.

On the other hand, the bacterial infection of the specific intraocular cavity such as the aphakic eye is presumably accelerated if (1) the amount of bacteria introduced into a corpus vitreum increases when the distanc between an iris and a lens vary; (2) bacteria can be proliferated in the cortex of the lens within the capsula lentis (which usually serves a strong barrier preventing bacteria from entering); and (3) the aqueous humor flow-passage from the anterior chamber is disturbed or the aqueous humor is disturbed or remains. Another type of aphakic eye (not a normal phakic eye) is presumably produced by introducing a viscoelastic substance. This is because the viscoelastic substance probably inhibits the normal self-cleansing activity of the aqueous humor, allowing bacterial infection [Oshida et al. IOVS, p S251, vol. 42, No. 4, 2001]. The present inventors clarified the on-set mechanism of the endophthalmitis as described above and achieved the present invention.

According to an aspect of the present invention, the viscoelastic substances used herein are roughly classified into two groups. One is a polymer viscoelastic substance which is excellent in preventing the space of a body cavity from collapsing. The other is a lower-molecular viscoelastic substance which is excellent in protecting the surface. Any viscoelastic substance may be used as long as it belongs to either one of the two groups.

According to an aspect of the present invention, any viscoelastic substance may be used as long as it is generally used in intraocular surgery and/or intra-articular injection. More specifically, such a viscoelastic substance may be hyaluronic acid, a non-toxic salt thereof, chondroitin sulfuric acid, a non-toxic salt thereof, hydroxypropyl methylcellulose (HPMC), or the like. According to an aspect of the present invention, the term “antimicrobial agent” used herein is a substance exhibiting an “antimicrobial effect” and an “antimycotic effect”. The antimicrobial agent may be a substance biologically produced or artificially synthesized. Examples of such an antimicrobial agent include

amino glycoside based antimicrobial agents such as streptomycin, dihydrostreptomycin, dihydrodeoxystreptomycin, fradiomycin, neomycin, paromomycin, aminocidine, kanamycin, kanamycin B, tobramycin, dibekacin, amikacin, gentamycin, micronomycin, ribostamycin, bekanamycin, and cisomysin;

tetracycline-based antimicrobial agents such as chlortetracycline, oxytetracycline, tetracycline, doxycycline, and minocycline;

chloramphenicol based anitmicrobial agents such as chloramphenicol and thiamphenicol;

macrolide based antimicrobial agents such as erythromycin, spiramycin, acetylspiramycin, midecamycin, leukomycin, kitasamycin, josamycin, and oleandomycin;

lincomycin based antimicrobial agents such as lincomycin and clindamycin;

penicillin based antimicrobial agents such as benzyl penicillin, phenoxymethylpenicillin, phenethicillin, propicillin, phenepenicillin, methicillin, oxacilin, cloxacilin, dicloxacillin, fulchroxacillin, ampicillin, amoxicillin, cyclacyllin, hetacillin, mecillinam, pivmecillinam, pivampicillin, talampicilin, bacampicilin, carbenicillin, carindacillin, carfecillin, ticarcillin, sulbenicillin, pyperacillin, apalcillin, and mezlocillin;

cephem-based antimicrobial agents such as cefalothin, cephaloridine, cefazolin, cephaloglycin, cephalexin, cefadroxil, cefatrizine, cefaclor, cefamandole, cefuroxime, cefotiam, cefoxitin, cefinetazol, cefsulodin, cefoperazone, ceftizoxime, cefotaxime, ceftazidine, cefixime, cefbuperazon, cefotetan, cefminox, latamoxef and flomoxef;

monobactam based antimicrobial agents such as aztreonam, and calmonum;

antimycosis agents such as nystatin, amphotericin B, trichomycin, pimaricin, griseofulvin, flucytosine, clotrimazole, miconazole, econazole, isoconazole, ketoconazole, fluconazole and itraconazole;

sulfur agents such as sulfathiazole, sulfamethizole, sulfisomidine, sulfamethoxsazol, sulfamethoxy pyrimidine, sulfamethoxin, and sulfaphenazole, sulfamonomethoxin;

sulfone agents such as 4,4′-diaminodiphenylsulfone, sodium glucosulfone, sodium sulfoxone, and thiazolsulfone;

antituberculosis agents such as paraaminosalicyl acid, isoniazid, ethionamide, polothionamide, and ethambutol;

quinolone-based antimicrobial agents such as nalidixic acid, pipemidic acid, ofloxacin, norfloxacin, enoxacin ciprofloxacin, tosfloxacin, levofloxacin, and sparfloxacin;

nitorofuran compounds such as nitrofurantoin, furazolidone, nifratel, nifraden, nitorofurazone, and nifuroxime;

dihydrofolic acid reductase inhibitors such as trimethoprim and pyrimethamine; and

general antimicrobial agents such as cycloserine, phophomycin, bacitracin, vancomycin, biomycin, capreomycin, fusidic acid, rifampicin, polymixin B, colistin, and gramicidin S.

According to an aspect of the present invention, examples of the anti-inflammatory agent used herein may include

steroid agents such as dexamethazone, fluorometholone, and betamethasone; and

nonsteroidal antipyretic and analgesic and antiphlogistic agents such as aspirin, mefenamic acid, indomethacin, indomethacin farnesyl, sulindac, acemethacin, dichlofenac sodium, mofezolac, nabumetone, tiaprofenic acid, oxaprozine, naproxen, pranoprofen, zaltprofen, ibuprofen, ketoprofen, loxoprofen sodium, flurbiprofen axetil, sulpyrine, tiaramide hydrochloride, acetaminophen, and azulene sulfonic acid.

According to an aspect of the present invention, the pharmaceutical composition of the present invention may be used in ophthalmological interocular surgery such as cataract, glaucoma, and vitreum surgery, orthopedic surgery accompanying intra-articulation injection for treating various articular disorders.

For example, in ophthalmological surgery, especially in cataract surgery, the surgery is successful if the anterior chamber is prevented from collapsing. When a closed cavity such as the eyeball is dissected, the aqueous humor flows out from the anterior chamber by inner pressure, and thereby the eyeball collapses. Since operation is performed within the intraocular space of the eyeball, the intraocular cavity, especially, the anterior chamber, the intraocular space must be protected from collapsing to attain successful surgery. As another technique of cataract surgery, an artificial lens is implanted within the eyeball. In this case, it is also important to create a space for a lens within the intraocular cavity (i.e., capsula lentis). To form the intraocular space, the pharmaceutical composition of the present invention is successfully used.

A technical idea of mixing a viscoelastic substance and an antimicrobial agent has been disclosed by Jpn. Pat. Appln. KOKAI Publication No. 9-208476 concerning a pharmaceutical composition for abdominal administration, and Jpn. Pat. Appln. KOKAI Publication No. 10-182390 concerning an oral composition. However, both Applications are concerned with the slow-release administration of an active ingredient from a viscoelastic substance in order to treat the portion infected with bacteria. Therefore, the invention recited in the claims of the present invention differs from these references in purpose and application site as well as effect to be produced.

Acta Ophthalmol (February, 1991, 69(1): 50-6) discloses intraocular treatment for endophthalmitis by a mixed agent of a viscoelastic substance and an antimicrobial agent. This paper teaches that the administration of an antimicrobial agent with the help of a viscoelastic substance is effective since the half-life of the antimicrobial agent can be extended. However, this technique differs from that of the present invention disclosed in this application. To reiterate, according to an aspect of the present invention, the pharmaceutical composition is directed to treating and preventing endophthalmitis. As far as we know, the use of a viscoelastic substance containing an antimicrobial agent or an anti-inflammatory agent in cataract surgery and a joint-space injection is neither disclosed nor shown as an example.

The present inventors found that it is useful to mix an antimicrobial agent into a viscoelastic substance to prevent bacterial infection in a closed cavity. They also found that it is extremely useful to add an anti-inflammatory agent as a medication of the pharmaceutical composition of the present invention in order to prevent inflammations associated with the bacterial infection or inflammations caused by bacterial invasion to tissue. Based on these findings, the present invention was accomplished.

Conventionally, a viscoelastic substance has been introduced alone to an aseptic body cavity such as the intraocular cavity or the joint space. An antimicrobial agent has been also introduced alone to such an aseptic body cavity by either systemically through oral intake or intravenous drip or locally through an ophthalmic solution or intraocular injection. Under these circumstances, an idea of using a pharmaceutical composition containing a viscoelastic substance and an antimicrobial agent or an anti-inflammatory agent in intraocular surgery and intra-articular injection is quite innovative. The use of such a pharmaceutical composition brings prominent effects. In other words, the pharmaceutical composition can effectively prevent infection possibly caused by the use of a viscoelastic substance and prevent inflammation associated with the infection. The pharmaceutical composition of the present invention has another advantage. The pharmaceutical composition of the present invention prevents bacteria from eluding physiological disinfection and sterilization in consequence of presence of a viscoelastic substance.

A closed body cavity is mostly contaminated with exogenous bacteria which enter from the outside at the time medical treatment or surgery is performed. However, the pharmaceutical composition of the present invention effectively works against infection and inflammation caused by not only the exogenous bacteria but also endogenous bacteria which migrate from other body part to the closed body cavity as is often seen at a poor immunization state.

According to an aspect of the present invention, the viscoelastic substance, antimicrobial agent, and anti-inflammatory agent to be used in the pharmaceutical composition of the present invention are not limited to the aforementioned agents. Accordingly, various modifications may be made without departing from the spirit or scope of the general inventive concept as defined by the appended claims and their equivalents.

The present invention will be explained with reference to the following examples, which are used as just an example and thus should not be construed as limiting the scope of the present invention.

### EXAMPLE 1

### Measurement for the Number of Living Bacterial Cells in the Presence of Viscoelastic Substance

<Method>

The proliferation rate of bacterial cells was checked in the presence of a viscoelastic substance of the present invention. The bacterial strains used herein were:

staphylococcus aureus MK99-3 (referred to as “S. aureus”), and

Stenotrophomonas maltophilia TK-1 (referred to as “S. maltophilia”).

Bacterial cells were cultured in a penicillin cup placed on an agar plate. After culturing, bacterial cells were added to physiological saline to prepare a strain solution having 0.5 McFarland (1.5×10⁸ CFU/ML). The bacterial cells were also added to a Muller-Hinton broth to prepare another strain solution having 0.5 McFarland. 0.1 mL of sodium hyaluronate agent, Healon^{R} (referred to as “HL”, and manufactured and sold by Pharmacia & Upjohn) was added in the penicillin cup and allowed to stand still and thereafter, 0.1 mL of each strain solution was added. This was designated as a first group. 0.1 mL of each strain solution was singly added to a penicillin cup. This was designated as a second group. The first and second groups were cultured in an incubator at 35° C. for 2, 4, 6 and 20 hours. A proliferation curve was obtained afterward.

<Results and Consideration>

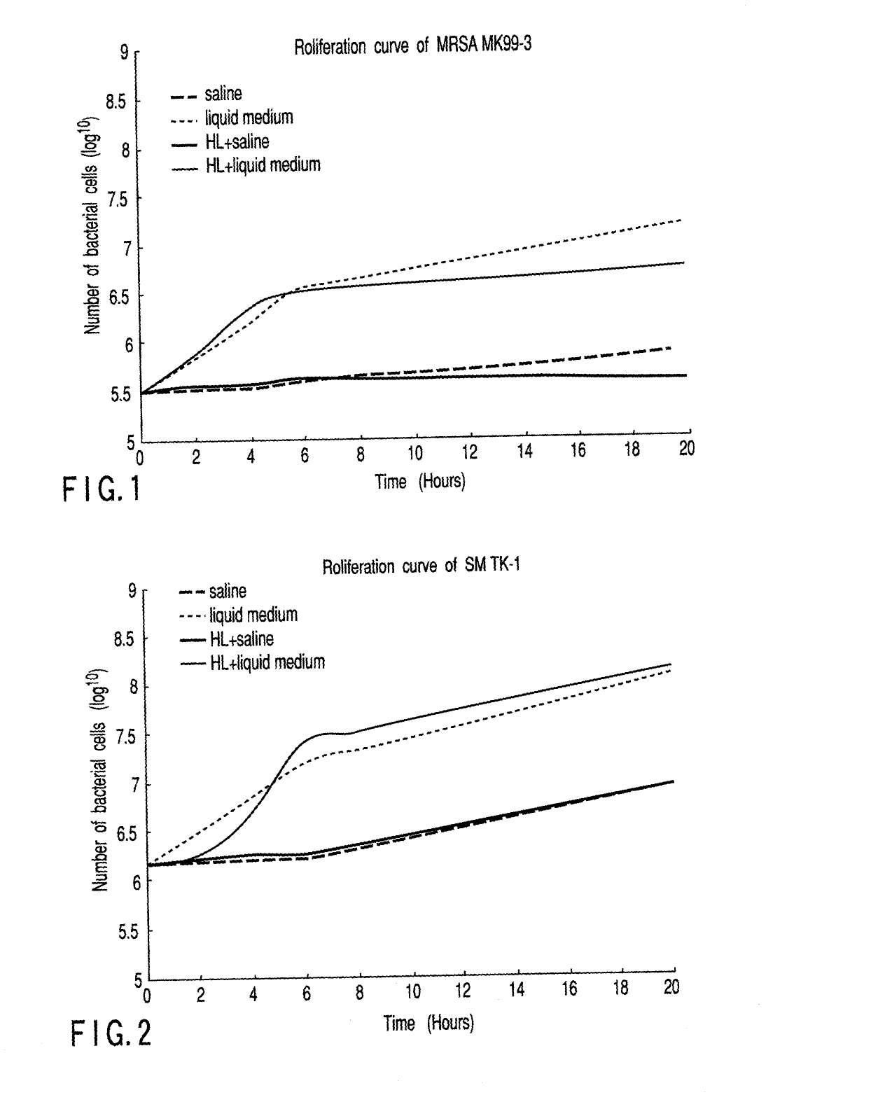

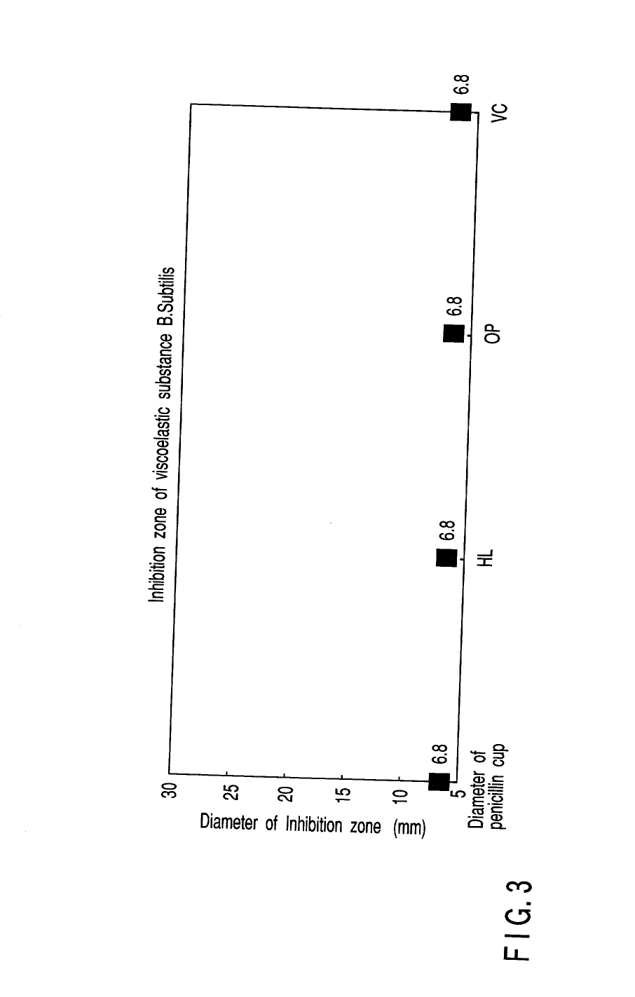

The bacterial cells in liquid broth proliferated more rapidly than those in physiological saline. The proliferation of S aureus is not so obvious in physiological saline, whereas the proliferation of S. maltophilia is significant. The bacterial cells in physiological saline and in liquid broth draw similar proliferation curves regardless the presence and absence of HL. This suggests that HL has neither proliferation accelerating activity nor proliferation suppressing activity with respect to S. aureus and S. maltophilia (See FIGS. 1 and 2).

Bacterial contamination of a viscoelastic substance has been reported in several papers. From the proliferation curve obtained in the experiments of the present invention, it is demonstrated that a viscoelastic substance may be contaminated with bacteria based on the fact that HL has neither bacterial proliferation accelerating activity nor proliferation suppressing activity. Although a high nutritional environment does not occur in normal conditions, bacterial growth would not take place. However, S. maltophilia can proliferate even in a low nutritional environment. Thus, care must be taken to S. maltophilia

### EXAMPLE 2

### Measurement of Antimicrobial Activity Changed by Viscoelastic Substance

<Method>

The activity of an antimicrobial agent changes depending upon a type of viscoelastic substance. Such a change was measured by using Bacillus substilis (ATCC6633 referred to as “B subtilis”) and the following viscoelastic substances:

Sodium hyaluronate agent (HL);

Opegan Hi^{R} (OP) manufactured by Santen Pharmaceutical Co., Ltd;

Viscoat^{R} (VS), a mixed agent of sodium hyaluronate and sodium chondroitin sulfuric acid manufactured by Alkon Laboratories Inc;

As antimicrobial agents, the following agents were used:

Crabit (LVFX) containing a levofloxacin manufactured by Santen Pharmaceutical Co., Ltd; and

Noflo (NFLX) containing norfloxacin, manufactured by Banyu pharmaceutical Co., Ltd.

First, B. subtilis (0.1 mL of 1.4×10⁸ CFU/ML) was spread on a 4% heart infusion medium. Subsequently, a penicillin cup was placed on the medium, and a test solution was poured in the cup.

As test solutions, the following groups were used.

1) Single Antimicrobial Agent Group Consisting of Solutions Each Containing an Antimicrobial Agent Alone

0.1 mL of an antimicrobial agent was added to a penicillin cup. Five concentrations from 10 μl/mL to 0.625 μg/L were prepared for each antimicrobial agent;

2) Single Viscoelastic Substance Group Consisting of Solutions Each Containing a Viscoelastic Substance Alone

0.1 mL of a viscoelastic substance was added to a penicillin cup;

3) Layered Group Consisting of Solutions Each Containing an Antimicrobial Agent and a Viscoelastic Substance in the Form of a Layer

After 0.1 mL of a viscoelastic substance was added to a penicillin cup and allowed to stand still and then 0.1 mL of 5 or 2.5 μg/mL antimicrobial agent was gently added to the viscoelastic substance layer; and

4) A mixture group consisting of solutions containing a viscoelastic substance and an antimicrobial agent in the form of a mixture

0.1 mL of a viscoelastic substance and 0.1 mL of 5 or 2.5 μg/mL antimicrobial agent were mixed and added to a penicillin cup.

Each group was cultured in an incubator at 35° C. for 24 hours.

The size of the inhibition circle of B. subtilis was measured. Based on the measurement results of the single antimicrobial agent group, a curve showing the size of inhibition circle versus the antimicrobial agent concentration (referred to as a standard curve) was obtained with respect to individual antimicrobial agents.

<Results and Consideration>

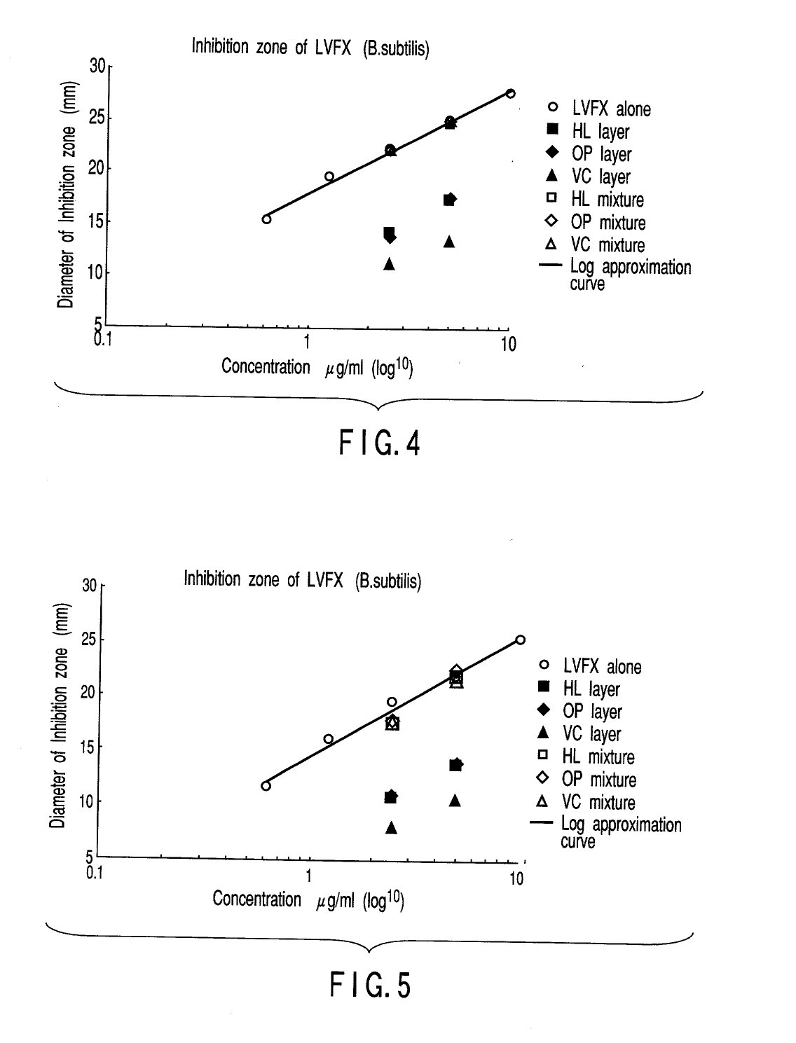

From the inhibition circle of the viscoelastic substances shown in FIG. 3, it is found that HL, OP, and VC exhibit no bacterial proliferation suppression activity against B. subtilis. When the results of the layered group (LVFX is layered on a viscoelastic substance) are compared to the standard curve of LVFX, the activity of the antimicrobial agent (LVFX) of any one of viscoelastic substances, HL, OP and VC is lower than the standard. In particular, the antimicrobial activity of VC is the lowest. In the mixture group of a viscoelastic substance and LVFX, the bacterial proliferation suppression activities of LVFX in the cases of HL, OP, and VC are lower than values of the standard curve but not so significant, as shown in FIG. 4.

When the results of the layered group (NFLX is layered on a viscoelastic substance) are compared to the standard curve of NFLX, the activity of the antimicrobial agent (NFLX) in any one of viscoelastic substances, HL, OP and VC is lower than the standard. In particular, the antimicrobial activity of VC is the lowest. In the mixture group of a viscoelastic substance and NFLX, the bacterial proliferation suppression activities of NFLX in the cases of HL, OP, and VC are lower than values of the standard curve but not so significant, as shown in FIG. 5.

In short, the formation of the inhibition circle by an antimicrobial agent is inhibited in the layered group. It is conceivable that a viscoelastic substance may trap an antimicrobial agent and inhibit it from locomotion.

Focusing upon viscoelastic substances, the size of the inhibition circle is then analyzed. The diameter of the inhibition circle is smaller than that of the standard in the cases of the layered groups using HL and OP. The size of the inhibition circle of VC is further smaller than the cases of Hl and OP. Such a difference may be caused by the difference in concentration, molecular weight, and viscosity between the viscoelastic substances. Further investigation is required on this matter. Conversely to say, this fact means that VC has an activity of protecting the endothelium. On the other hand, the formation of the inhibition circle by an antimicrobial agent is not inhibited in the mixture groups of HL, OP and VC. From the aforementioned results, it is presumed that the activity of the antimicrobial agent is not suppressed chemically but physically due to the presence of a layer.

In bacterial infection, bacteria must deposit on a tissue before they start proliferation. Once bacteria deposit, they form a biofilm. Because of the form of a film, it becomes difficult to remove the bacteria. Actually, bacteria enter the eyeball even though it is sterilized by an antimicrobial agent during ophthalmologic surgery. To suppress the bacteria thus introduced, an antimicrobial ophthalmic solution is applied after surgery. However, when a viscoelastic substance is used, it difficult to completely remove the viscoelastic substance used in surgery from the eye. In addition, a viscoelastic substance likely to remain in the eye has been put in practical use. With the use of viscoelastic substance, a risk of bacterial deposition may increase. The bacterial deposition is considered to be overcome by allowing an antimicrobial agent to locomotion from a viscoelastic substance. Also, the use of viscoelastic substance may have more character, concerning the clearance activity of the aqueous humor from the anterior chamber and an action of avoiding unnecessary immune attack.

According to the studies of Oshida et al. (Japan Ophthalmologic Society, p 102, vol. 105, 2001) and the present inventors (Tanaka et al., p S251, vol. 42, No. 4, 2001), bacteria given to the anterior chamber are quickly removed and, completely removed from the aqueous humor of the anterior chamber after 24 hours. They reported that even though bacterial deposition and proliferation are pathologically observed in the endothelium of the cornea and the surface of the iris, no bacteria is seen in a cultured sample of the aqueous humor of the anterior chamber. It is considered that the bacterial growth may be prevented by the clearance activity of the aqueous humor and leucocytes phagocytosis.

In this experiment, it is demonstrated that the efficacy of an antimicrobial agent can be completely maintained by previously mixing it with a viscoelastic substance. Since a viscoelastic substance is just mixed with a microbial agent and therefore does not prevent the microbial agent from releasing, bacterial deposition can be successfully prevented by the antimicrobial agent. Furthermore, the following effects:

the clearance activity of the aqueous humor; and

the activity of a viscoelastic substance for preventing bacterial deposition by covering over bacteria, thereby avoiding unnecessary immune attack can be expected.

The present invention is efficiently used as a slow-release administration method of a medication to the eyeball. More specifically, in the present invention, an antimicrobial agent is added to a viscoelastic substance, which may remain within the intraocular surface for about 2 to 4 hours after surgery and administrated into a body cavity. Therefore, the antimicrobial agent can act at the most requisite time and site.

It is believed that such a viscoelastic substance is required more and more in future. Accordingly, a further development and technical advance will be demanded. Under these circumstances and from the prophylaxis point of view, it is desirable to add an antimicrobial activity to a viscoelastic substance, in addition to the activities of protecting a space from collapsing and protecting the endothelium.

Now, the composition of the present invention will be explained by way of examples, which are limited to the specific details shown and described herein and will not construe the scope of the present.

### EXAMPLE 3

### Composition

1. Intraocular Composition

A viscoelastic substance has been widely used during cataract surgery to protect the anterior chamber from collapsing and to form the posterior capsule (posterior chamber) as well as to protect the endothelium of the cornea. The viscoelastic substance is indispensably used in cataract surgery presently performed all over the world. The cost of such a viscoelastic substance is covered by health insurance in Japan.

In the intraocular surgery including cataract surgery, many complications occur, the most significant being an infection such as endophthalmitis. When such an infection occurs, an antimicrobial agent is externally administered through an ophthalmic solution or an intraocularly through injection. Alternatively, surgery is performed by widening the operation area to include the vitreum. However, the eyesight after the surgery becomes worse than before. Although conventional viscoelastic substances, which have molecular weights within the range of 10 thousands to 20 millions, have been used alone, any one of them may be used in couple with a medication in the present invention.

According to an aspect of the present invention, a pharmaceutical composition is prepared by adding an antimicrobial agent or an anti-inflammatory agent to a viscoelastic substance in a concentration within the conventional range of concentration and used in cataract surgery. The concentration of an antimicrobial agent to be added to a viscoelastic substance desirably falls within the range effectively working in the eyeball. Now, two examples of intraocular compositions will be described below.

<Composition 1>

| 1 mL of Composition 1 contains | |
|---|---|
| Sodium hyaluronate: | 30 mg |
| Sodium chondroitin sulfuric acid: | 40 mg |
| Levofloxacin | 21 μg |

The concentrations of components of Composition 1 are determined with reference to the concentrations of a commercially available viscoelastic substance, Viscoat in ophthalmologic surgery and Crabit ophthalmic solution (manufactured Santen Pharmaceutical Co., Ltd.). Each of the concentrations shown above is a concentration effectively working in the eye. As to the concentration if levofloxacin effectively working in the eye, see “Pharmacological activity of levofloxacin ophthalmic solution and intraocular behavior” Antibiotics & Chemotherapy, Vol. 16, No. 8, 76-84 (2000)”.

<Composition 2>

| 1 ml of Composition 2 contains | |
|---|---|
| Sodium hyaluronate: | 100 mg |
| Ofloxacin | 2 μg |

This is used to prevent infections such as post-cataract surgery endophthalmitis.

2. Articular Composition

In articular disorders such as articular osteoarthritis (gonarthritis), periarthritis, and chronic rheumatoidarthritis, arthralgia is a matter of a great concern since it causes ADL (activity of daily living) disorder. Arthralgia is caused by destruction of articular cartilage. Therefore, a viscoelastic substance is injected into a joint space to mitigate such a pain. This treatment has been performed worldwide. The injection of a viscoelastic substance may cause bacterial contamination of a joint space that has been inherently aseptic. The infection of the joint space with bacteria induces suppurative arthritis. Once suppurative arthritis happens, even if an antimicrobial agent is systemically or locally administered, the prognosis is not good, leading to a severe functional disorder of the articulation. This is the worst case an orthopedic surgeon is afraid of. It is expected that such a microbial infection of the joint may be inhibited by the mixture of an antimicrobial agent and a viscoelastic substance.

Furthermore, when a viscoelastic substance is injected into the joint space, the inflammation of the synovial membrane of the joint occurs due to a side effect. The mixture of an antimicrobial agent and a viscoelastic substance is also considered to be useful in preventing the inflammation of the synovial membrane of the articulation.

Although conventional viscoelastic substances, which have molecular weights within the range of 10 thousands to 20 millions, have been used alone, any one of them may be used in couple with a medication in the present invention.

According to an aspect of the present invention, a pharmaceutical composition is prepared by adding an antimicrobial agent or an anti-inflammatory agent to a viscoelastic substance in a concentration with in the conventional range and used in articular injection.

Now, examples of an articular composition will be shown below.

<Composition 3>

| 1 mL of composition contains | |
|---|---|
| Sodium hyaluronate: | 25 mg |
| Lincomycin hydrochloride | 500 mg |

Composition 3 of the present invention is prepared by mixing the components listed above. The pharmaceutical composition can prevent a post-operative infection such as arthritis.

According to the present invention, it is possible to prevent and/or treat an infection caused by bacterial contamination at the time a viscoelastic substance is injected. Furthermore, it is possible to prevent to bacterial deposition and proliferation at the time a viscoelastic substance is singly applied to a body cavity. Moreover, when an antimicrobial agent is administered systemically through oral intake or an intravenous drip or locally through an ophthalmic solution, usually an antimicrobial-resistant strain is developed. However, in the present invention, since an antimicrobial agent is administered to a body site in a small amount by way of a viscoelastic substance, the development of the antimicrobial-resistant strain can be prevented.

According to the present invention, inflammations caused by surgery using a viscoelastic substance can be prevented and/or treated.

The references used herein the entire contents of which are incorporated in the specification by reference.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A pharmaceutical composition containing a medication and a viscoelastic substance.

2. A pharmaceutical composition according to claim 1 for use in intraocular surgery.

3. A pharmaceutical composition according to claim 1 for introduction into an articular cavity.

4. A pharmaceutical composition according to any one of claims 1 to 3, wherein said viscoelastic substance is selected from the group consisting of hyaluronic acid, nontoxic salts thereof, chondroitin sulfuric acid, nontoxic salts thereof, hydroxypropyl methylcellulose and a mixture of these compounds.

5. A pharmaceutical composition according to any one of claims 1 to 3, wherein said medication is selected from the group consisting of anti-inflammatory agents and antimicrobial agents.

6. A pharmaceutical composition according to any one of claims 1 to 3, wherein said medication is an antimicrobial agent and said a viscoelastic substance is a mixture of sodium hyaluronate and chondroitin sulfuric acid.
